(19) United States
(12) Patent Application Publication
(10) Pub. No.: US 2006/0266358 A1
(43) Pub. Date: Nov. 30, 2006

(54) Hand held bellows resuscitator

(76) Inventor: Christopher Hoogland, Woodstock, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/439,740

(22) Filed: May 24, 2006

Related U.S. Application Data
(60) Provisional application No. 60/684,059, filed on May 24, 2005.

(51) Int. Cl. A61M 16/00

(52) U.S. Cl. 128/205.13; 128/205.14; 128/204.28

(57) ABSTRACT

An improved hand operated resuscitator is provided. In place of a squeeze bag for pumping air or oxygen into the face mask, this resuscitator has a bellows for the hand pumping of air or oxygen. In one embodiment the resuscitator is provided with a limiting mechanism that prevents the full expansion of the bellows so that the amount of air or oxygen delivered to the patient can be limited. This limiting mechanism can be a strap with a fastener that encircles the bellows of the resuscitator. It may be adjusted to provide the optimal amount of air of oxygen to the patient. Another embodiment has a stop mechanism that that prevents the full compression of the bellows. This stop mechanism may be a lever and a physical stop.

### CLAIM TO PRIORITY

The present application claims the benefit of the filing of U.S. Provisional Patent Application Ser. No. 60/684,059 filed on May 24, 2005, which is hereby incorporated by reference herein.

### BACKGROUND OF THE INVENTION

1. Field of the Invention

This invention relates to a hand held resuscitator that uses a bellows for pumping the air or oxygen to the face mask or airway tube.

A resuscitator, or breathing equipment, is adapted to supply ambient air, or oxygen from a pressurized source, to a person to aid in their breathing. The resuscitator has a bellows, which can be adjusted to supply more or less air or oxygen to the patient, a mask to be applied over the mouth and nose or to a tube that goes directly into the trachea (endotracheal tube or combitube or similar airway tube) of the patient, and a valve interconnecting the mask and the bellows. The valve mechanism prevents gases that are exhaled from the patient's lungs from being returned to his lungs upon the next succeeding inspiration.

2. Prior Art

A hand-operated resuscitator is described in U.S. Pat. No. 4,077,404 (Elam). Elam describes a hand operated resuscitator with an automatic valve mechanism, which prevents the gases that are exhaled from the patient's lungs from being returned to his lungs upon the next succeeding inspiration. The resuscitator disclosed in this patent has a manual squeeze bag that can be squeezed by the person administering air or oxygen to a person to aid in his breathing. Elam describes in detail the valve mechanism for a resuscitator. Elam's resuscitator uses a ball valve that moves back-and-forth along a cylinder between an inspiration port and valve seat and an expiration port and valve seat on the opposite end. When the ball closes the inspiration port, an exhalation channel is provided to the atmosphere. When the ball closes the expiration port, an inspiration channel is provided to a side wall port. The squeeze bag is provided to pump air through the inspiration port.

U.S. Pat. No. 4,374,521 (Nelson et al.) discloses a squeeze bag type resuscitator. Nelson's resuscitator is designed to administer oxygen from a pressurized source. Their resuscitator has a demand responsive oxygen supply valve along with a manually compressible squeeze bag. A demand supply valve is mounted on, and communicates with the end of the squeeze bag opposite the end on which the face mask is located. The squeeze bag resuscitator has a manually compressible and self-restoring bag. The interior of the bag is coupled to the face mask. The operator manually squeezing the bag forces air into the patient's lungs. When the operator releases the squeezing pressure on the bag, the bag restores itself due to the resiliency of the bag. This fills the bag with either fresh atmospheric air or oxygen which allows repeating the cycle. The squeeze bag is designed to be held and squeezed with one hand by the operator. A check valve permits refilling the bag with air or oxygen during its restoration without removing the mask from the face of the patient.

The squeeze bag of Nelson et al. is formed by molding a synthetic resin plastic or rubber material. The squeeze bag has a hollow central section and a frusto-conical end section on each end with a central opening. The apparatus is intended to be held with one hand of the operator, with the squeeze bag being squeezed by that hand, when oxygen or air is needed. When the operator withdraws pressure from the squeeze bag, the bag by virtue of its resilient and self-restoring nature restores itself to its normal state.

U.S. Pat. No. 4,774,941 (Cook) discloses a resuscitator having a squeeze bag with a gas inlet and a gas outlet, a first valve with a squeeze bag port, a patient port and an exhalation port. This first valve allows “forced respiration,” “free exhalation” and “spontaneous breathing.” Cook discloses a conventional squeeze bag. The bag has a gas outlet and gas inlet.

One of the problems with the resuscitators that are currently available is that the manual squeeze bag is hard to squeeze for many people, especially people with small hands. The size of the squeeze bag and is its cylindrical shape make it difficult for many people to squeeze with one hand. Frequently the operator has to hold the mask or do other tasks and has only one hand free to squeeze the bag.

It is an object of this invention to develop a way for an operator to be able to supply air or oxygen to a patient with one hand. It is a further object of this invention to develop a squeezeable chamber that can be easily squeezed by one hand, even of a small person. It is a further object of this invention to develop a air chamber where the amount of air supplied to the patient can be easily controlled. Less air should be provided to a child or small adult. Excess air can damage the lungs of a person.

### SUMMARY OF THE INVENTION

This invention replaces the squeeze bag of a resuscitator for supplying air or oxygen to the face mask or other airway tube with a bellows, which basically consists of a bag-like structure with sides and pleats between the sides. The bellows is hinged at one end with a supply pipe for supplying air or oxygen to the face mask or other airway tube. At least one side of the bellows is hinged and can be moved back-and-forth in respect to the other side of the bellows. An inlet vent is provided so that air can enter the bellows when it is expanded. This inlet vent has a valve to prevent air from escaping from the bellows when the sides of the bellows are compressed against each other. An outlet vent is provided to the supply pipe so that air or oxygen is pushed into the supply pipe when one side of the bellows is compressed against the other. This outlet vent also has a valve to prevent air or oxygen entering the bellows from the supply pipe. In other words, this outlet valve is a one way valve that only permits air or oxygen to enter the supply pipe. The inlet valve is also a one way valve that only permits air or oxygen to enter the bellows. Thus, when the bellows is compressed by pushing one side against the other, any air or gas in the bellows is pushed into the supply pipe. When the bellows is open, which occurs by allowing one side to expand against the other side of the bellows, air or oxygen will enter the bellows through the one way inlet valve.

Unlike with a squeeze bag, the bellows can be easily operated with one hand by even a small person, because of the leverage provided between the end of each side of the bellows and the point at which the bellows is hinged to the supply pipe.

The bellows can be provided with a stop mechanism so that it does not become fully compressed. When the bellows are fully expanded, the end (distal end) of each side remote from the hinge point would be at the extreme distance between the two sides. A stop can be provided so that the bellows can only be compressed part way. When the bellows is only compressed part way, a smaller amount of air or gas would be pushed into the supply pipe to go to the face mask or tube for the patient. This is important because in many cases as a lesser amount of air or oxygen should only be administered to the patient. The stop mechanism can also have a number of positions so that the precise amount of air or oxygen being administered to the patient is precisely controlled by the operator to the level desired.

Alternatively, the bellows can be provided with a mechanism, such as a strap to prevent the bellows from opening to the fully open position. This will insure that a smaller amount of air will be provided to the patient. The mechanism can have several different partially open positions for patients of different sizes. It may have positions for different sizes of children and small adults. A strap can be provided to extend around both sides of the bellows to prevent the bellows from being fully opened. This strap can be constructed like a belt with a buckle and holes to set different bellow positions.

### BRIEF DESCRIPTION OF THE DRAWINGS

FIG. 1 is a side elevation view of the resuscitator of this invention which shows the bellows.

FIGS. 2A-2B are schematic side views of the bellows of this invention showing the expanded mode (FIG. 2A) and the compressed mode (FIG. 2B). FIG. 2A illustrates an embodiment with a stop to prevent the bellows from being fully compressed when the stop is in the stop up position.

FIGS. 3A-3B are schematic side views of the bellows of the invention in the compressed mode.

FIGS. 4A-4B are schematic views of the bellows of this invention showing the hinge and hinged side and stationary side of the bellows.

FIG. 5 is a schematic view showing the supply tube and hinge of the hinged bellows.

FIGS. 6A-6B are schematic views of one embodiment of this invention showing a stop mechanism for preventing the bellows from being completely compressed.

FIGS. 7A-7B are schematic views of another embodiment of this invention showing a stop mechanism that has a lever for stopping the bellows from being fully compressed.

FIGS. 8A-8E are schematic views of another embodiment of this invention showing a mechanism, in this case a strap, that prevents the bellows from being fully opened.

FIG. 8E is a view of the strap of this invention showing different positions for fastening the strap.

### DETAILED DESCRIPTION OF THE DRAWINGS

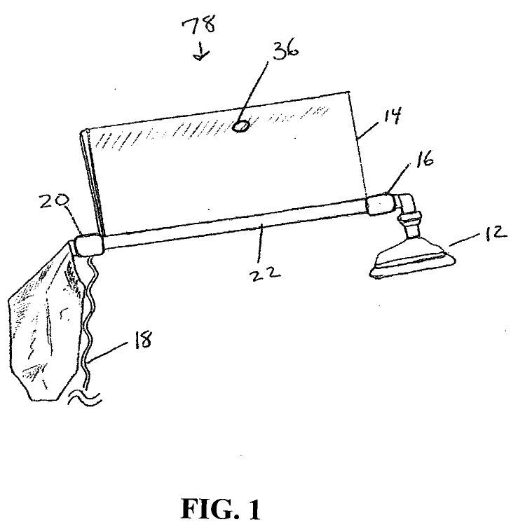

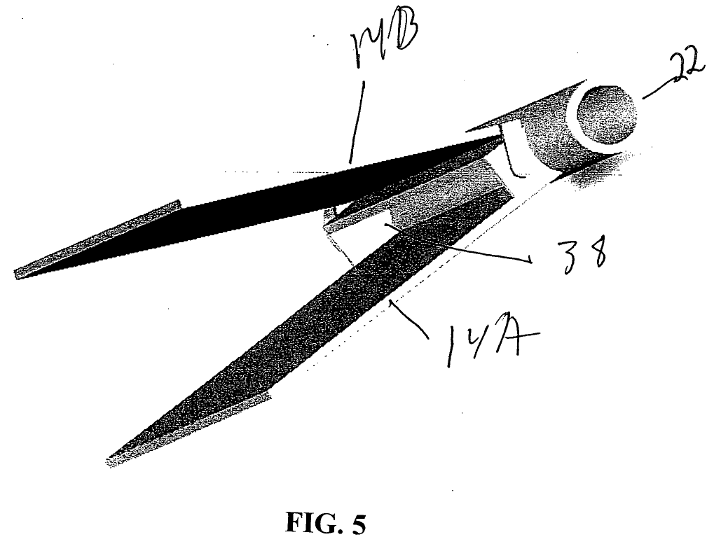

The bellows resuscitator of this invention is illustrated in FIG. 1. The resuscitator is designed to be operated by a person with one hand. The resuscitator 10 has a face mask 12 into which air or oxygen is pumped by a bellows 14. The bellows 14 pumps air or oxygen into a supply tube 22 through a one way outlet valve 38 as shown in FIG. 5. The oxygen or air flows through to a patient valve mechanism 16 for entry into the face mask 12. The resuscitator has an oxygen supply tube 18 connected to a source of oxygen. The resuscitator 10 also has a demand supply valve 20.

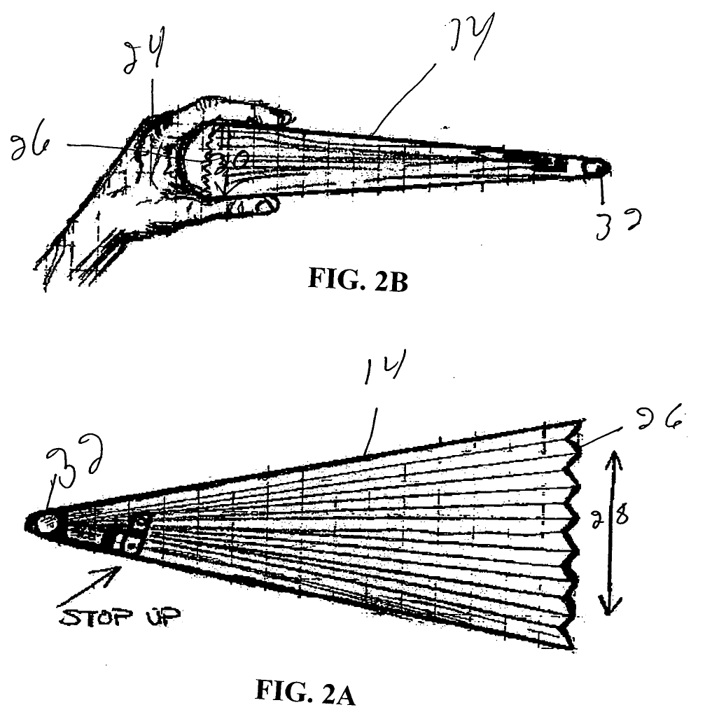

As shown in FIGS. 2A and 5 the bellows 14 is pleated with a stationary side 14A and a hinged side 14B. The bellows 14 has a bellows inlet valve 36 which is a one way valve allowing air or oxygen to enter into the bellows as it is being expanded. The bellows 14 has a bellows outlet valve 38 which is a one way valve only allowing air or oxygen to enter into the supply tube 22 for delivery to the patient.

As illustrated in FIGS. 2A-2B the bellows 14 can be operated by one hand 24. Considerable leverage is available as a hand 24 squeezes the distal end 26 of the bellows 14 which is far removed from the hinge point 32 of the bellows. Air or oxygen enters through the bellows inlet valve 36 when the bellows 14 is expanding and the air or oxygen exits through the bellows outlet valve 38 into the supply tube 22 when the bellows 14 is compressed.

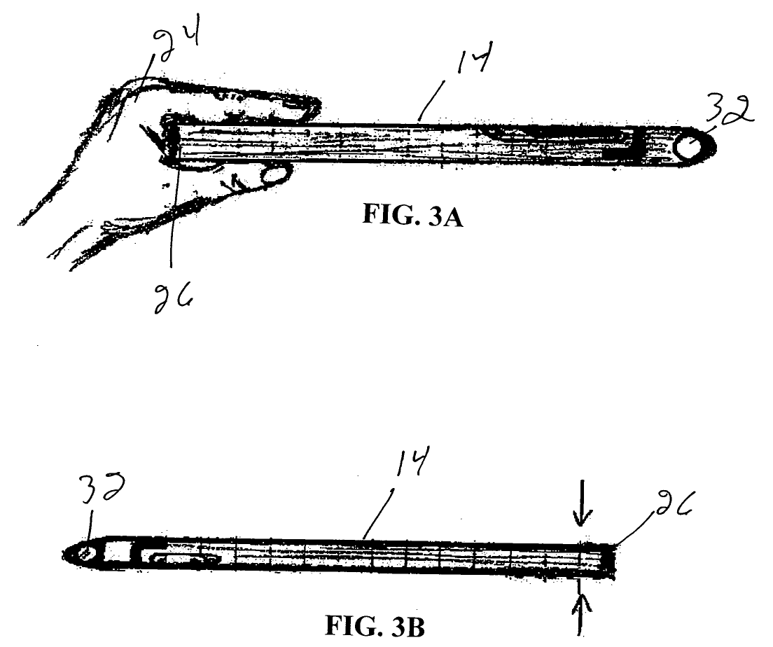

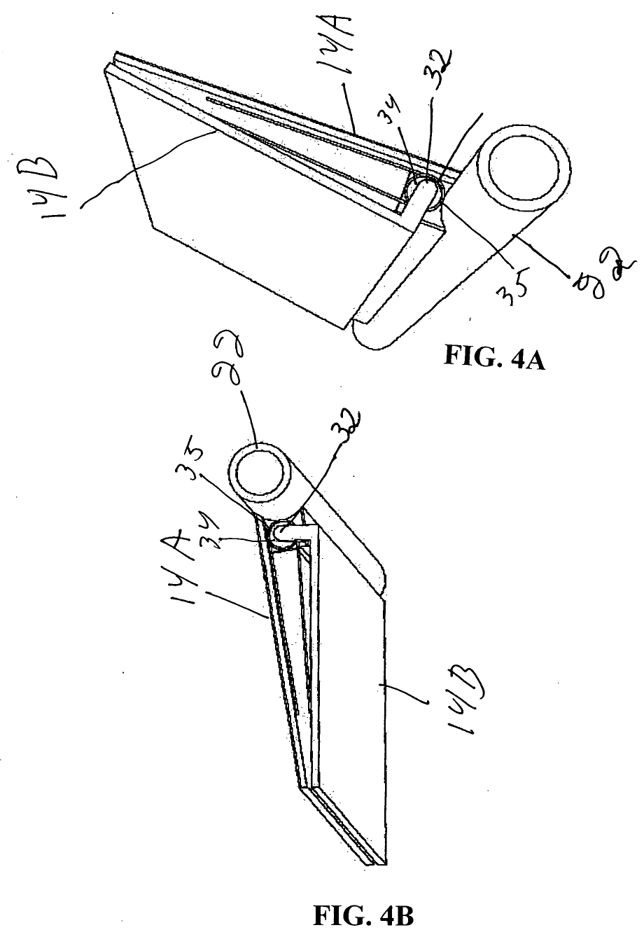

The compression of the bellows 14 is shown in FIGS. 3A and 3B. The hinging of the bellows 14 is shown in FIGS. 4A and 4B which are schematic figures. It is shown that only the hinged side 14B of the bellows 14 moves when the bellows is compressed and expanded. In this case the hinge point 32 is basically a shaft 34 that extends through a tube 35. Other types of hinges can be provided for allowing the compression and expansion of the bellows 14. A schematic view of the inside of the bellows is shown in FIG. 5 which shows the outlet valve 38.

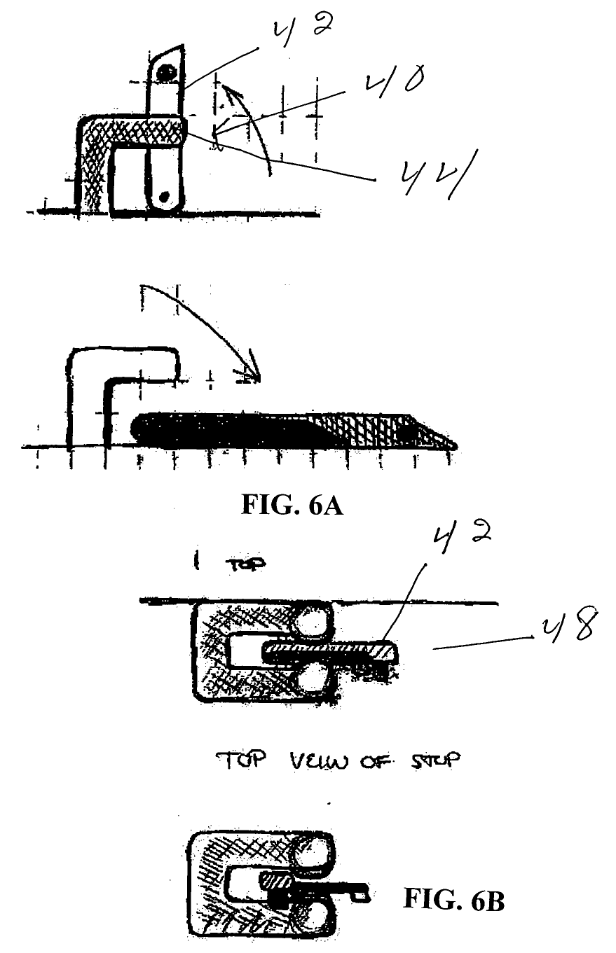

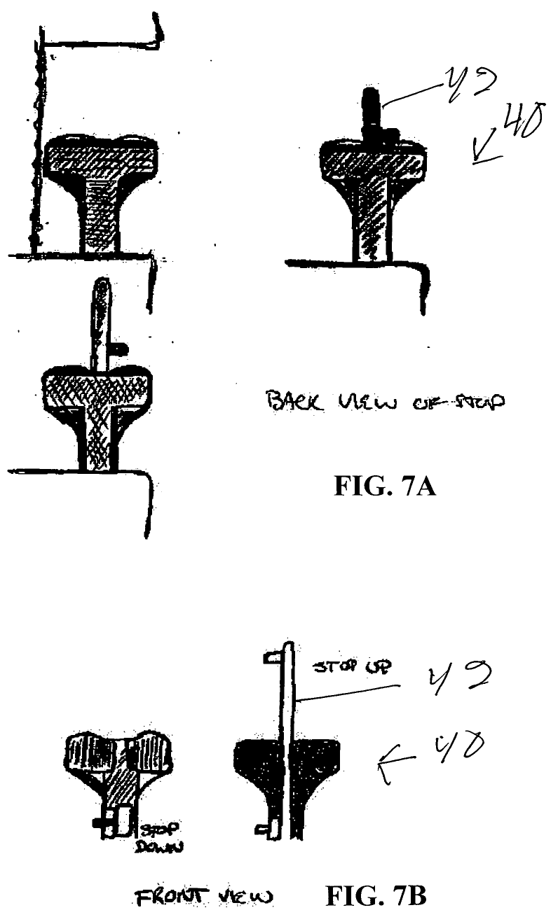

It is frequently desirable to only pump a limited amount of air or oxygen into a patient's lungs to prevent the lungs from rupturing. The bellows 14 may have a stop 40 as shown in FIGS. 6A and 6B so that it can only be compressed so far. This is important in order to allow the supply of a limited amount of air or oxygen to a patient as may be a requirement for a child or certain types of adult patients, such as small adults. In this stop mechanism 40 a stop lever 42 is provided. The stop lever 42 is simply rotated around a hinge point 44 into the stop up position where it engages the bellows 14 as illustrated in FIG. 2A to prevent the bellows from being compressed to the fully compressed state. This stop lever 42 can be rotated into the stop down position as shown in FIG. 6B in order to allow a full bellows of air or oxygen to be pumped into the patient's lungs. The rotating of this stop mechanism 40 is illustrated in FIGS. 7A and 7B.

While a simple hinged stop lever is illustrated in FIGS. 6A and 6B, it should be realized that other type of stop mechanisms may be used to prevent the full compression of the bellows. For example a more sophisticated type of stop mechanism could include a dial that the operator could set to indicate the percentage of compression desired (e.g. 50 percent). This dial could be connected to a stop lever 42 as shown in FIGS. 6A and 6B to only allow limited compression of the bellows. Other types of stop mechanisms, either non-adjustable or adjustable, can be utilized for this invention. Another type of stop mechanism prevents the bellows from opening too far. In a typical approach the stop mechanism would have three settings with one for a child, one for a small adult and one for a normal or large adult.

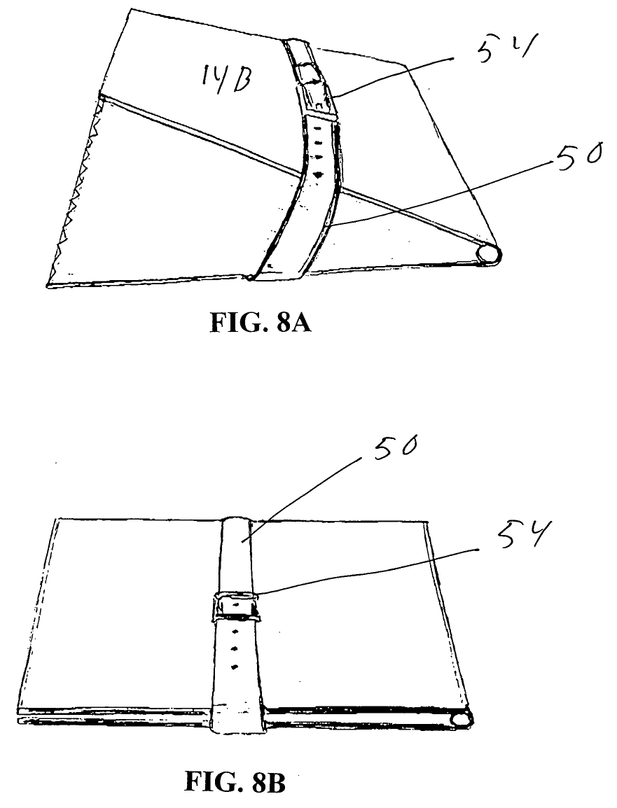

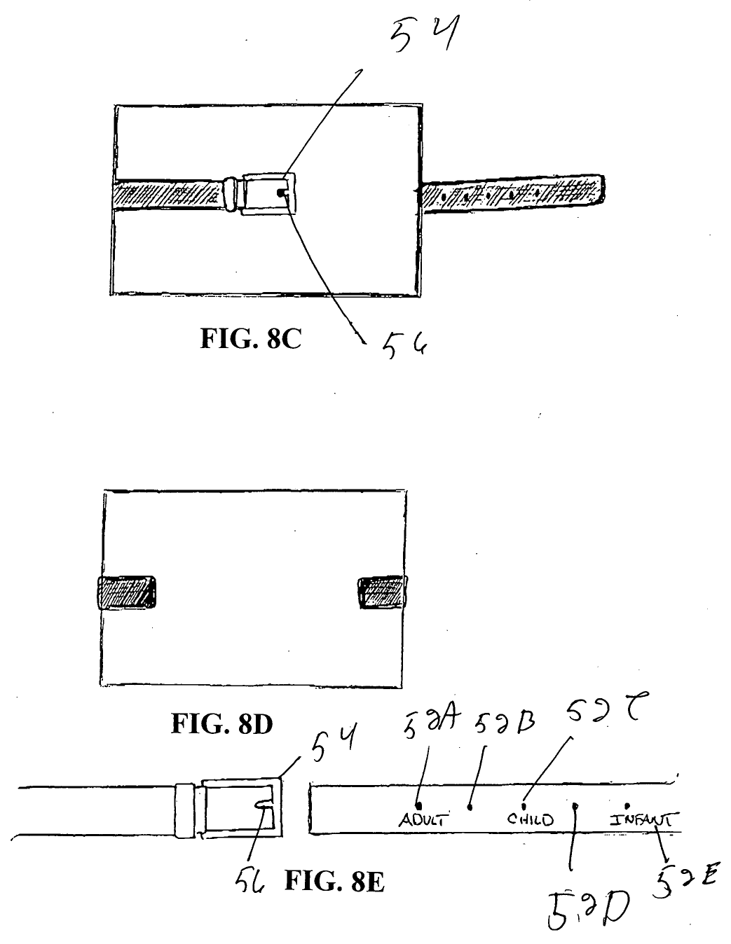

In another embodiment of this invention as shown in FIGS. 8A- 8E, a strap 50 is used to prevent the bellows 14 from opening too far. The strap extends around the bellows 14. The strap can have a fastening mechanism, for setting the position to which the bellows 14 can be expanded to allow the intake of air or oxygen. In this case, the strap 50 is constructed like an adjustable belt. Holes 52A-52E can be punched in the strap 50. The strap 50 may have a buckle 54 with a tongue 56 that may be extended through a hole 52A-52E to lock the strap 50 in position.

With this feature the resuscitator may be used with individuals with different lung capacity. With an infant for example the strap 50 would be set at position 52E before the bellows 14 is opened for compression. In this way only a limited amount of air or oxygen could be pumped into the lungs, thus preventing lung rupture.

It should be realized other types of mechanisms can be used to prevent the bellows from being completely closed or with a number of partially open settings.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

Therefore, having thus described the invention, at least the following is claimed:

1. In a hand operated resuscitator having a face mask or outlet tube, and a supply tube for supplying oxygen or air to the face mask or outlet tube, the improvement being a hand pumped bellows for supplying air or oxygen to the supply tube.

2. The resuscitator of claim 1, in which the bellows has a one way air inlet valve for allowing air or oxygen into the bellows when it is in its expanding and expanded states and a one way air outlet valve for supplying air or oxygen to the supply tube as the bellows is compressed.

3. The resuscitator of claim 1, in which a stop mechanism is provided for preventing the full compression of the bellows so that only a portion of the capacity of the bellows is used for supplying air or oxygen to the supply tube.

4. The resuscitator of claim 2, in which a stop mechanism is provided for preventing the full compression of the bellows so that only a portion of the capacity of the bellows is used for supplying air or oxygen to the supply tube.

5. The resuscitator of claim 1 which also has a stop mechanism which can be set to only allow the partial compression of the bellows, said stop mechanism comprising a hinged lever and a stop, with the hinged lever being capable of moving around the stop from an open to a stop position, so that the bellows can be fully compressed when the hinged lever is in the open position and only partially compressed when the hinged lever is in the stop position.

6. The resuscitator of claim 2 which also has a stop mechanism which can be set to only allow the partial compression of the bellows, said stop mechanism comprising a hinged lever and a stop, with the hinged lever being capable of moving around the stop from an open to a stop position, so that the bellows can be fully compressed when the hinged lever is in the open position and only partially compressed when the hinged lever is in the stop position.

7. The resuscitator of claim 1 which also has a limiting mechanism that can be set to prevent the bellows from being opened to the fully expanded position.

8. The resuscitator of claim 2 which also has a limiting mechanism that can be set to prevent the bellows from being opened to the fully expanded position.

9. The resuscitator of claim 7 in which the limiting mechanism is a strap with a fastener that encircles the bellows and is capable of being fastened to prevent the bellows from being opened to the fully expanded position.

10. The resuscitator of claim 8 in which the limiting mechanism is a strap with a fastener that encircles the bellows and is capable of being fastened to prevent the bellows from being opened to the fully expanded position.

11. A hand operated resuscitator comprising a face mask or outlet tube, a supply tube for supplying air or oxygen to the face mask or outlet tube, a bellows for supplying air or oxygen to the supply tube, an inlet valve for permitting the passage of air or oxygen into the bellows.

12. The resuscitator of claim 11, in which the bellows has a one way air inlet valve for allowing air or oxygen into the bellows when it is in its expanding and expanded states and a one way air outlet valve for supplying air or oxygen to the supply tube as the bellows is compressed.

13. The resuscitator of claim 11 which also has a limiting mechanism that can be set to prevent the bellows from being opened to the fully expanded position.

14. The resuscitator of claim 12 which also has a limiting mechanism that can be set to prevent the bellows from being opened to the fully expanded position.

15. The resuscitator of claim 13 in which the limiting mechanism is a strap with a fastener that encircles the bellows and is capable of being fastened to prevent the bellows from being opened to the fully expanded position.

16. The resuscitator of claim 14 in which the limiting mechanism is a strap with a fastener that encircles the bellows and is capable of being fastened to prevent the bellows from being opened to the fully expanded position.
